(19) United States
(12) Patent Application Publication
(10) Pub. No.: US 2009/0005706 A1
(43) Pub. Date: Jan. 1, 2009

(54) Medical guide wire

(75) Inventors: Naohiko Miyata, Aichi-ken (JP); Satoshi Nagano, Aichi-ken (JP); Tadahiro Koike, Aichi-ken (JP)

Correspondence Address: MORGAN LEWIS & BOCKIUS LLP, 1111 PENNSYLVANIA AVENUE NW, WASHINGTON, DC, 20004, US

(73) Assignee: Asahi Intecc Co., Ltd, Aichi-Ken (JP)

(21) Appl. No.: 12/213,475

(22) Filed: Jun. 19, 2008

(30) Foreign Application Priority Data
Jun. 22, 2007 (JP) 2007-164836

(51) Int. Cl. A61M 25/01

(52) U.S. Cl. 600/585

(57) ABSTRACT

A medical guide wire 1 has a front helical spring 4 which is formed by two stranded wire units 41, 42 in a helically-wired spring configuration in which the two stranded wire units 41, 42 are further twisted in a helically-wired multiple spring configuration. This imparts an increased flexibility to the front helical spring 4, compared to the case in which a front helical spring is formed by a single wire to be diametrically identical to the front helical spring 4. With the favorable flexibility and sufficient torque-transmissibility, it is possible to reduce a risk of having a damage on a blood vessel upon inserting the medical guide wire 1 into the blood vessel.

The present invention claims priority to Japanese Patent Application No. 2007-164836 filed Jun. 22, 2007, the disclosure of which is incorporated herein by reference in its entirety.

### BACKGROUND OF THE INVENTION

1. Field of the Invention

The invention relates to a medical guide wire well-suited to a medical usage, and particularly concerns to a medical guide wire used upon inserting a catheter into a blood vessel, urethra and organs, and also used upon leading a retainer to an aneurysm-forming tract area.

2. Description of Prior Art

In a medical guide wire, a flexibility is required for a distal end of the guide wire not to have a damage on a blood vessel, while at the same time, a sufficient torque-transmissibility is required to transmit a manual operation from a proximal side to a distal end of the guide wire.

In order to satisfy the above requirements, a core shaft is prepared, a distal end portion of which is thinned and a rear end of the core shaft is thickened. The core shaft is fixed to a helical spring body with the core shaft inserted into the helical spring body.

The helical spring body is formed by bonding a front helical spring to a basal helical spring, the former of which is made of a single wire. This type of the guide wire is disclosed by Japanese Laid-open Patent Application Nos. 2005-46603, 2006-271955, Japanese Patent Publication No. 4-25024 and Japanese Patent No. 2737650.

Japanese Laid-open Patent Application No. 2005-46603 discloses a guide wire which has a coiled spring provided to enclose a distal end portion of core shaft. A fixing member is provided to concentrically secure the coiled spring to the core shaft, so that the distal end of the guide wire does not partially deform upon steering the guide wire through the somatic cavity.

Japanese Laid-open Patent Application No. 2006-271955 discloses a guide wire in which a core shaft has a projection axially extended in a concentrical relationship with the core shaft. A coiled spring is concentrically provided to enclose a distal end portion of the guide wire, and bonded to the projection, so that the distal end of the guide wire does not partially deform upon inserting the guide wire into the somatic cavity.

Japanese Patent Publication No. 4-25024 discloses a guide wire having a flexible tubular core made of a high torsion metal. A first coiled spring made of a metallic material, and secured to a distal end portion of the tubular core. A second coiled spring made of a radiopaque material, and connected to a distal end of the first coiled spring so as to exhibit a high flexibilty and good torque-transmissibility upon inserting the guide wire into the cardiovascular tract.

Japanese Patent No. 2737650 discloses a coiled connector, one end of which is screwed to a distal helical spring, and the other end of which is screwed to a basal helical spring. This enables manufacturers to connect the distal helical spring to the basal helical spring without sacrificing a good flexibility at a portion in which the distal helical spring is connected to the basal helical spring.

In the prior art guide wires, when a distal end portion of the guide wire is thinned to decrease its diameter so as to make it more flexible, the thinned distal end portion decreases the torque-transmissibility, thus making it difficult to transmit a basal side operation to the distal end portion of the guide wire.

Therefore, it is an object of the invention to overcome the above drawbacks so as to provide a medical guide wire which is capable of enhancing a flexibility of a front helical spring while insuring a sufficient torque-transmissibility.

### SUMMARY OF THE INVENTION

According to the present invention, a medical guide wire is provided to have a core shaft, a distal end side of which is thinned to have a reduced diameter, and a proximal end side of which is thickened to have an increased diameter. A helical spring body has a front helical spring provided at a distal side of the helical spring body and a basal helical spring provided at a rear side of the front helical spring. The core shaft and the helical spring body are fixed together with the distal end side of the core shaft inserted into the helical spring body. The front helical spring has stranded wires formed by a plurality of wires stranded in a helically-wired spring configuration. The basal helical spring is formed by a single wire in a helically-wired spring configuration.

With the front helical spring formed by the stranded wires upon assembling the guide wire, it is possible to make the guide wire more flexible without thinning the core shaft while insuring a sufficient torque-transmissibility.

According to the other aspect of the present invention, the front helical spring has a stranded wire unit formed by stranding three to nineteen wires in the helically-wired spring configuration.

According to the other aspect of the present invention, the front helical spring has the stranded wire units counted as three to nineteen. These stranded wire units are further stranded to forms the helically-wired multiple spring configuration.

With the helically-wired multiple spring configuration, it is possible to enhance a mechanical strength, compared to the case in which one stranded wire unit is formed in the helically-wired spring configuration.

According to the other aspect of the present invention, the stranded wire units are made of a metallic wire selected from the group consisting of stainless steel wire, Ni—Ti alloyed wire and radiopaque wire, otherwise the stranded wire units are made of at least two kinds of metallic wires selected from the group consisting of stainless steel wire, Ni—Ti alloyed wire and radiopaque wire.

According to the other aspect of the present invention, the front helical spring uses two stranded wire units to form the helically-wired multiple spring configuration, and one of the two stranded wire units is made of a metallic wire selected from the group consisting of stainless steel wire, Ni—Ti alloyed wire and radiopaque wire, and the other hand, the rest of the two stranded wire units is made of at least two kinds of metallic wires selected from the group consisting of stainless steel wire, Ni—Ti alloyed wire and radiopaque wire.

According to the other aspect of the present invention, one of a rear end of the front helical spring and a forward end of the basal helical spring is diametrically reduced integrally to have a diameter-reduced end, so that the diameter-reduced end is inserted into the other of the rear end of the front helical spring and the forward end of the basal helical spring to connect the front helical spring to the basal helical spring.

As a prior art method of bonding the front helical spring to the basal helical spring, the front helical spring is meshed with the basal helical spring to be screwed to the basal helical spring by means of soldering. Otherwise, a helical connector is separately prepared, one end of which is meshed with the front helical spring to be screwed to the front helical spring, and other end of which is meshed with the basal helical spring to be screwed to the basal helical spring.

Both of the connecting methods, however, require complicated procedures in which the front helical spring, the basal helical spring and the helical connector are meshed each other to be screwed, thus rendering the assemble procedures time-consuming.

Contrary to the prior art methods, according to the invention, it suffices to insert the diameter-reduced end of the front helical spring or the basal helical spring upon bonding the former to the latter, thus making it possible to render the assemble procedures simple and quick.

With the diameter-reduced end integrally formed on the front helical spring or the basal helical spring, it is possible to make whole the assemble simple without increasing the number of the necessary component parts.

According to the other aspect of the present invention, a helical connector is provided which has an outer diameter slightly smaller than inner diameters of both the rear end of the front helical spring and the forward end of the basal helical spring. A front end portion of the helical connector is inserted into the rear end of the front helical spring, and a rear end portion of the helical connector is inserted into the forward end of the basal helical spring.

The helical connector makes it simple to insert the helical connector to the front helical spring and the basal helical spring upon bonding the former to the latter.

### BRIEF DESCRIPTION OF THE DRAWINGS

Preferred forms of the present invention are illustrated in the accompanying drawings in which:

FIG. 1 is a longitudinal cross sectional view a medical guide wire according to a first embodiment of the invention but partly sectioned;

FIG. 2 is an enlarged longitudinal cross sectional view designated by a circle E of FIG. 1;

FIG. 3 is an enlarged longitudinal cross sectional view of a basal helical spring;

FIG. 4 is a longitudinal cross sectional view of a main portion of the medical guide wire according to a second embodiment of the invention; and

FIG. 5 is a longitudinal cross sectional view of a main portion of the medical guide wire according to a third embodiment of the invention.

### DETAILED DESCRIPTION OF PREFERRED EMBODIMENTS OF THE INVENTION

In the following description of the depicted embodiments, the same reference numerals are used for features of the same type.

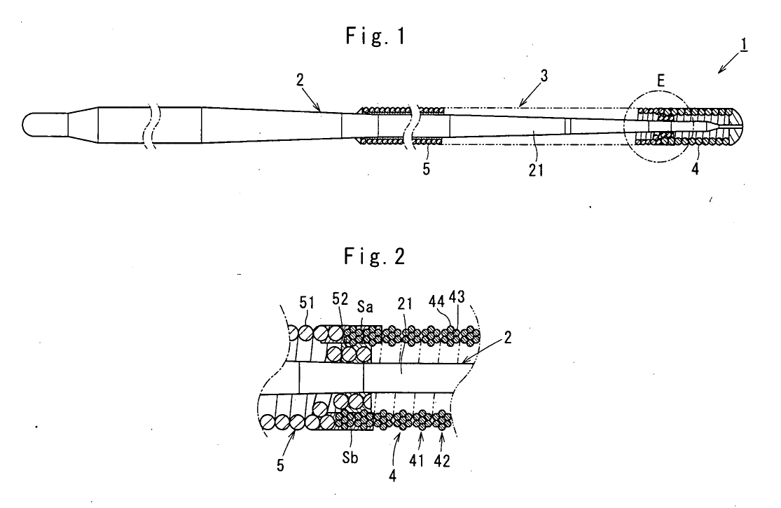

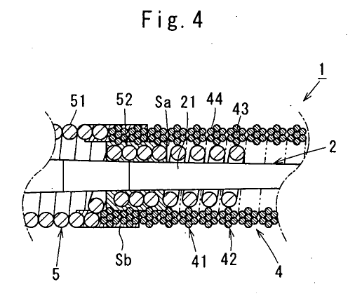

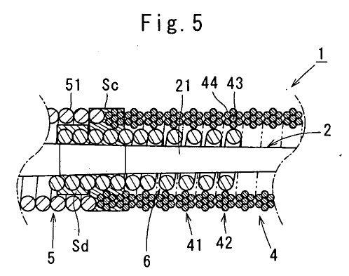

Referring to FIGS. 1 through 3, described is a medical guide wire 1 according to a first embodiment of the invention. In FIGS. 1 through 3, a right hand side stands a distal end side and a left hand side represents a proximal or rear end side. The same is true with second and third embodiments in FIGS. 4 and 5.

The medical guide wire 1 (shortened as “guide wire 1” hereinafter) has a core shaft 2 and a helical spring body 3. The helical spring body 3 is secured to the core shaft 2 with a distal end portion 21 of the core shaft 2 inserted into the helical spring body 3.

The core shaft 2 is made of stainless steel, Ti—Ni based alloy or the equivalents. The core shaft 2 has a distal end portion thinned to have a decreased diameter and having a rear end portion thickened to have an increased diameter.

The helical spring body 3 has a front helical spring 4 and a basal helical spring 5 provided at a rear end of the front helical spring 4.

The front helical spring 4 is formed by two stranded wire units 41, 42 which are arranged consecutively in a helically-wired multiple spring configuration. Each of the stranded wire units 41, 42 has seven wire elements consisting of one core wire 43 and six side wires 44. The six side wires 44 are stranded around the core wire 43.

The core wire 43 is made of stainless steel, and the side wires 44 are made of radiopaque material (e.g., Pt—Ni based alloy).

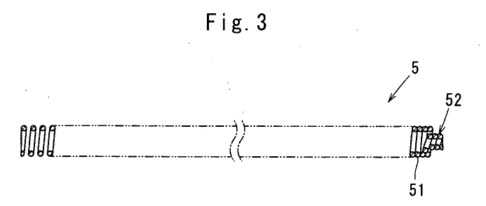

The basal helical spring 5 is formed by a single wire 51 in a helically-wired spring configuration. A diameter of the forward end of the basal helical spring 5 is diametrically smaller than that of the rear portion of the basal helical spring 5 which integrally forms a diameter-reduced end 52 as shown in FIG. 3.

An outer diameter of the diameter-reduced end 52 is slightly smaller than an inner diameter of a rear end of the front helical spring 4. The basal helical spring 5 inserts its diameter-reduced end 52 into the rear end of the front helical spring 4 with a permissible clearance presented therebetween. The diameter-reduced end 52 is soldered to the core shaft 2, while at the same time, the diameter-reduced end 52, the front helical spring 4 and the basal helical spring 5 are fittingly bonded by means of soldering Sa, Sb. It is to be noted that the basal helical spring 5 is made of a high rigidity material such as, for example, stainless steel or cobalt-based alloy. On the outer surface of the helical spring body 3, a hydrophilic layer is coated, although not shown.

By way of illustration, the helical spring body 3 is 200 mm long, the front helical spring 4 is 30 mm long, and the basal helical spring 5 is 170 mm long. The core wire 43 and the side wire 44 of the stranded wire units 41, 42 have an outer diameter of 0.025 mm. An outer diameter of the front helical spring 4 measures 0.33 mm, and an inner diameter of the front helical spring 4 measures 0.18 mm.

An outer diameter of the single wire 51 measures 0.05 mm. An outer diameter of the basal helical spring 5 measures 0.33 mm, and an inner diameter of the basal helical spring 5 measures 0.23 mm. An outer diameter of the diameter-reduced end 52 measures 0.17 mm with its length as 0.5 mm.

With the front helical spring 4 formed by the stranded wire units 41, 42, it is possible to make the guide wire more flexible, while at the same time, insuring a sufficient torque-transmissibility, compared to the case in which a front helical spring is formed by a single wire to be diametrically identical to the front helical spring 4. With the flexibility insured by the front helical spring 4,

it is possible to reduce a risk of having a damage on the blood vessel, so as to safely implement the vascular treatment therapeutically.

With the front helical spring 4 formed by the stranded wire units 41, 42, it is possible to increase a rupture-resistance against a torsional deformation, compared to the case in which the front helical spring is formed by the single wire. This is also preferable to safely implement the vascular treatment.

With the diameter-reduced end 52 inserted into the rear end of the front helical spring 4 upon bonding the basal helical spring 5 to the front helical spring 4, needed is such a simple and quick procedure as to insert the diameter-reduced end 52 into the front helical spring 4.

With the diameter-reduced end 52 soldered to the front helical spring 4, it is possible to insure a sufficient bonding strength between the diameter-reduced end 52 and the front helical spring 4 without resorting to the case in which the two helical springs are meshed and screwed to be bonded together by means of the soldering procedure.

With the diameter-reduced end 52 integrally formed with the basal helical spring 5, it is possible to assemble the guide wire 1 without increasing the number of the necessary component parts.

Even if an excessive tensile force disconnectedly breaks the distal end portion 21 of the core shaft 2, the tensile force deforms the front helical spring 4 to diametrically shrink around a remained portion of the core shaft 2 so as to develop a considerable amount of frictional resistance between an inner surface of the front helical spring 4 and an outer surface of the core shaft 2. The frictional resistance counters the tensile force working at a bonded portion between the front helical spring 4 and the basal helical spring 5. This helps to avoid the disconnection between the front helical spring 4 and the basal helical spring 5 so as to contribute to safely implementing the vascular treatment with the guide wire 1.

FIG. 4 shows a second embodiment of the invention in which the diameter-reduced end 52 is expanded along the core shaft 2 toward the distal side of the guide wire 1. The diameter-reduced end 52 is expanded in a tensile direction to broaden the helical pitch until the diameter-reduced end 52 reaches 10 mm in length from the original length (6 mm). The broadened helical pitch makes the diameter-reduced end 52 more flexible without excessively increasing the rigidity at the bonded portion between front helical spring 4 and basal helical spring 5, while at the same time, insuring a good restitutive capability.

FIG. 5 shows a third embodiment of the invention in which a helical connector 6 is discretely provided. The guide wire 1 has the core shaft 2, the helical spring body 3 and the helical connector 6. The helical connector 6 has an outer diameter slightly smaller than an inner diameter of the rear end of the front helical spring 4 and an inner diameter of the forward end of the basal helical spring 5.

A front end of the helical connector 6 is fittingly inserted into the rear end of the front helical spring 4, and a rear end of the helical connector 6 inserted into the forward end of the basal helical spring 5 so as to connect the front helical spring 4 to the basal helical spring 5 via the helical connector 6. The front helical spring 4, the basal helical spring 5 and the helical connector 6 are bonded together by means of solders Sc, Sd. The helical connector 6 is also bonded to the core shaft 2 by means of the solders Sc, Sd.

Upon connecting the front helical spring 4 to the basal helical spring 5, needed are such simple and quick procedures as to insert the helical connector 6 to the front helical spring 4 and the basal helical spring 5.

The helical connector 6 makes the complicated prior procedures unnecessary in which one helical spring is meshed with another helical spring to screw the former to the latter.

### Modification Forms

(A) The front helical spring 4 is formed by stranding the six side wires 44 around one core wire 43, however, the number of the wire elements for the front helical spring 4 is not confined only to seven.

The number of the wire elements for the stranded wire units 41, 42 may be within the range of three to nineteen. The number of the wire elements for the stranded wire unit 41 may differ from the number of the wire elements for the stranded wire unit 42.

(B) Instead of the two stranded wire units 41, 42, only one stranded wire unit may be used, otherwise more than three stranded wire units may be employed, although the two stranded wire units 41, 42 are preferable to preserve the flexibility and mechanical strength.
(C). Instead of the core wire 43 made of the stainless steel and the side wires 44 made of the radiopaque wire, the core wire 43 and the side wires 44 may be made from the same material such as the stainless steel, Ni—Ti based alloy or radiopaque material.
(D) In lieu of the core wire 43 made of the stainless steel and the side wire 44 made of the radiopaque material, the combination of core wire 43 and the side wire 44 may be made from at least two different kinds of material selected from the group consisting of the stainless steel, Ni—Ti based alloy and radiopaque material.
(E) The stranded wire unit 41 may be made from one metal selected from the group consisting of the stainless steel, Ni—Ti based alloy and radiopaque material, and the stranded wire unit 42 may be made from at least two different kinds of material selected from the group consisting of the stainless steel, Ni—Ti based alloy and radiopaque material.
(F) In place of the diameter-reduced end 52 integrally formed on the forward end of the basal helical spring 5, the diameter-reduced end 52 may be formed on the rear end of the front helical spring 4 to be inserted into the forward end of the basal helical spring 5 to connect the front helical spring 4 to the basal helical spring 5.
(G) Otherwise, the front helical spring 4 may be meshed with the basal helical spring 5 to be screwed to the basal helical spring 5, and the former is fittingly bonded to the latter by means of soldering procedure.
(H) Alternatively, a discrete helical connector may be provided, one end of which is screwed to respective one end of the front helical spring, and the other end of which is screwed to one end of the basal helical spring upon connecting the front helical spring to the basal helical spring.
(I) Although particular preferred embodiments of the present invention have been disclosed in detail for illustrative purposes, it will be recognized that variations or modifications of the disclosed apparatus, including the rearrangement of part, lie within the scope of the present invention.

What is claimed is:

1. A medical guide wire having a core shaft, a distal end side of which is thinned to have a reduced diameter, and a proximal end side of which is thickened to have an increased diameter,
said medical guide wire comprising:
a helical spring body having a front helical spring provided at a distal side of said helical spring body and a basal helical spring provided at a rear side of said front helical spring;
said core shaft and said helical spring body being fixed together with said distal end side of said core shaft inserted into said helical spring body; and
said front helical spring having stranded wires formed by a plurality of wires stranded in a helically-wired spring configuration, and said basal helical spring formed by a single wire in a helically-wired spring configuration.

2. The medical guide wire according to claim 1, wherein said front helical spring having a stranded wire unit formed by stranding three to nineteen wires in said helically-wired spring configuration.

3. The medical guide wire according to claim 1 or 2, wherein said front helical spring has a plurality of said stranded wire units further stranded to be formed in helically-wired multiple spring configuration.

4. The medical guide wire according to claim 1 or 2, wherein said stranded wire units are made of a metallic wire selected from the group consisting of stainless steel wire, Ni—Ti alloyed wire and radiopaque wire, otherwise said stranded wire units are made of at least two kinds of metallic wires selected from the group consisting of stainless steel wire, Ni—Ti alloyed wire and radiopaque wire.

5. The medical guide wire according to claim 3, wherein said front helical spring uses two stranded wire units to form said helically-wired multiple spring configuration, and one of said two stranded wire units is made of a metallic wire selected from the group consisting of stainless steel wire, Ni—Ti alloyed wire and radiopaque wire, and the rest of said two stranded wire units is made of at least two kinds of metallic wires selected from the group consisting of stainless steel wire, Ni—Ti alloyed wire and radiopaque wire.

6. The medical guide wire according to claim 1, wherein one of a rear end of said front helical spring and a forward end of said basal helical spring is diametrically reduced integrally to have a diameter-reduced end, so that said diameter-reduced end is inserted into the other of said rear end of said front helical spring and said forward end of said basal helical spring.

7. The medical guide wire according to claim 1, wherein a helical connector is provided which has an outer diameter slightly smaller than inner diameters of both said rear end of said front helical spring and said forward end of said basal helical spring, and a front end portion of said helical connector is inserted into said rear end of said front helical spring, and a rear end portion of said helical connector is inserted into said forward end of said basal helical spring.
